(19) United States
(12) Patent Application Publication
(10) Pub. No.: US 2026/0069340 A1
(43) Pub. Date: Mar. 12, 2026

(54) CRYOGENIC SURGICAL INSTRUMENT WITH RIGHT ANGLE END EFFECTOR

(71) Applicants: Salvatore PRIVITERA, Mason, OH (US); Christopher William WIDENHOUSE, Mason, OH (US); Matthew MONTI, Mason, OH (US); Kathe POCKER, Mason, OH (US); Tyler DAY, Mason, OH (US)

(72) Inventors: Salvatore PRIVITERA, Mason, OH (US); Christopher William WIDENHOUSE, Mason, OH (US); Matthew MONTI, Mason, OH (US); Kathe POCKER, Mason, OH (US); Tyler DAY, Mason, OH (US)

(21) Appl. No.: 19/391,772

(22) Filed: Nov. 17, 2025

Related U.S. Application Data
(63) Continuation-in-part of application No. PCT/US2024/034309, filed on Jun. 17, 2024.
(60) Provisional application No. 63/508,676, filed on Jun. 16, 2023.
(60) Provisional application No. 63/508,683, filed on Jun. 16, 2023.
(60) Provisional application No. 63/562,885, filed on Mar. 8, 2024.
(60) Provisional application No. 63/914,918, filed on Nov. 10, 2025.

(51) Int. Cl. A61B 18/02; A61B 18/00

(52) U.S. Cl. CPC A61B 18/02; A61B 2018/00101; A61B 2018/00172; A61B 2018/00434; A61B 2018/0225; A61B 2018/0262

(57) ABSTRACT

A cryogenic probe is disclosed herein, the cryogenic probe comprising a handle, a shaft comprising a proximal end coupled to the handle, wherein the shaft defines a longitudinal axis; an end effector coupled to a distal end of the shaft, wherein the end effector comprises a first prong, a second prong, and a curved section therebetween, wherein the first prong and the second prong are configured to receive cryogenic fluid, wherein the first prong and the second prong extend perpendicular to the longitudinal axis and are spaced apart along the longitudinal axis; and a supply conduit configured to supply cryogenic fluid to the end effector.

### CROSS-REFERENCE TO RELATED APPLICATIONS

This application is a continuation-in-part of PCT Application No. PCT/US2024/034309, filed Jun. 17, 2024, which claims priority to U.S. Provisional Applications No. 63/508,676 filed Jun. 16, 2023, No. 63/508,683 filed Jun. 16, 2023, and No. 63/562,885 filed Mar. 8, 2024, the entirety of which are incorporated by reference. This application also claims priority to U.S. Provisional Application No. 63/914,918, filed Nov. 10, 2025, the entirety of which is incorporated by reference.

### FIELD OF THE INVENTION

The present disclosure is directed to cryogenic surgical devices, and more specifically, to cryogenic surgical instruments with a cryogenic active zone and related methods.

### BACKGROUND OF THE INVENTION

Below knee amputation (BKA) is defined as any amputation above the ankle and below the knee. There are currently no widely practiced, non-pharmaceutical pain relief methods and devices thereof for limb amputation patients. This is partially due to the nerves in the leg comprising different sizes and being positioned at different locations, making it difficult for current devices to facilitate freezing for all potential nerves in the leg. Phantom limb pain (PLP) is the perception of pain that originates from an amputated limb. BKA affects about 60% of amputees, while PLP affects about 60%-85% of amputees.

About 185,000 people have an amputation each year in the United States. During amputation, pain relief can be achieved by freezing nerves at or near the amputation site. Surgeons generally freeze the nerve proximally while under visualization post-nerve cut and post-leg cut. However, current devices are unable to freeze nerves at desired locations due to the inability to grasp the nerves consistently.

Therefore, there exists a need for methods and devices to perform pain relief for BKA procedures with simple engagement of nerves by direct contact with multiple nerves at a time. The devices and methods should be able to freeze various surfaces of the nerves without the need for repeated freezes.

### SUMMARY OF THE INVENTION

In some aspects, the techniques described herein relate to a cryogenic probe, including, a handle; a shaft including a proximal end coupled to the handle, wherein the shaft defines a longitudinal axis; an end effector coupled to a distal end of the shaft, wherein the end effector includes a first prong, a second prong, and a curved section therebetween, wherein the first prong and the second prong are configured to receive cryogenic fluid, wherein the first prong and the second prong extend perpendicular to the longitudinal axis and are spaced apart along the longitudinal axis; and a supply conduit configured to supply cryogenic fluid to the end effector.

In some aspects, the techniques described herein relate to a cryogenic probe, further including an exhaust conduit configured to exhaust cryogenic fluid from the end effector.

In some aspects, the techniques described herein relate to a cryogenic probe, wherein the exhaust conduit is concentrically disposed around the supply conduit.

In some aspects, the techniques described herein relate to a cryogenic probe, further including a vacuum insulating layer disposed around the supply conduit.

In some aspects, the techniques described herein relate to a cryogenic probe, further including a tubular thermal barrier element disposed within the vacuum insulating layer.

In some aspects, the techniques described herein relate to a cryogenic probe, wherein the thermal barrier element is constructed of at least one of a ceramic, a fiberglass, an elastomer, a foam, silicon, carbon fiber, and a composite.

In some aspects, the techniques described herein relate to a cryogenic probe, wherein the end effector is inserted at the distal end of the shaft.

In some aspects, the techniques described herein relate to a cryogenic probe, wherein the end effector is coupled to the distal end of the shaft via a screw thread.

In some aspects, the techniques described herein relate to a cryogenic probe, further including a nozzle at a distal end of the supply conduit.

In some aspects, the techniques described herein relate to a cryogenic probe, wherein the nozzle terminates proximal to the curved section.

In some aspects, the techniques described herein relate to a cryogenic probe, wherein the end effector includes a prong base, wherein the prong base includes a constant outer diameter that transitions to the second prong.

In some aspects, the techniques described herein relate to a cryogenic probe, wherein a cross-section of the first prong and a cross-section of the second prong are rectangular.

In some aspects, the techniques described herein relate to a method for freezing one or more nerves of a patient, the method including: advancing a cryogenic probe to a target site, the cryogenic probe including a handle, a shaft including a proximal end coupled to the handle, and an end effector coupled to a distal end of the shaft, wherein the end effector includes a first prong, a second prong, and a curved section therebetween, wherein the first prong and the second prong are configured to receive cryogenic fluid, wherein the first prong and the second prong extend perpendicular to a longitudinal axis of the shaft and are spaced apart along the longitudinal axis; positioning the end effector to grasp one or more nerves; supplying cryogenic fluid from a supply conduit to the end effector; and freezing the one or more nerves by applying the end effector to surface of the one or more nerves.

In some aspects, the techniques described herein relate to a method, further including exhausting cryogenic fluid from the end effector via an exhaust conduit.

In some aspects, the techniques described herein relate to a method, further including a nozzle within the supply conduit.

In some aspects, the techniques described herein relate to a method, wherein the nozzle terminates at the curved section.

In some aspects, the techniques described herein relate to a method, wherein the nozzle terminates proximal to the curved section.

In some aspects, the techniques described herein relate to a medical device, including, a handle; a shaft including a proximal end coupled to the handle, wherein the shaft defines a longitudinal axis; an end effector coupled to a distal end of the shaft, wherein the end effector includes a first prong, a second prong, and a prong base, wherein the first prong and the second prong are configured to receive fluid, wherein the first prong and the second prong extend at an angle with respect to the longitudinal axis, wherein the prong base includes a cavity to receive the shaft; and a supply conduit configured to supply fluid to the end effector.

In some aspects, the techniques described herein relate to a medical device, wherein the fluid is a cryogenic fluid.

In some aspects, the techniques described herein relate to a medical device, further including a nozzle at a distal end of the supply conduit.

### BRIEF DESCRIPTION OF DRAWINGS

FIG. 1A illustrates a cryogenic probe in accordance with one variation of the invention.

FIG. 1B illustrates a close-up view of the distal end portion of the shaft of the cryogenic probe.

FIGS. 2A and 2B illustrate an end effector in accordance with one variation of the invention.

FIGS. 3A to C illustrate variations of cryogenic probes with varying nozzle positions.

FIGS. 4A to G illustrate variations of end effectors to be used with the cryogenic probe.

FIG. 5A illustrates another variation of the device having a nozzle that extends to a longitudinal distance where the second prong is located.

FIG. 5B illustrates a prong base of the end effector of FIG. 5A.

FIGS. 6A and 6B illustrate a variation of the end effector wherein the end effector comprises a neck portion between the second prong and the prong base.

FIGS. 7A to 7C illustrate a device having a handle, a first jaw, a second jaw, and a hinge according to one variation of the invention.

FIGS. 8A to 8C illustrate a system having a device, an inlet tube, and an exhaust tube according to one variation of the invention.

### DETAILED DESCRIPTION

The present disclosure contemplates that cryogenic surgical devices, such as surgical instruments with an active cryogenic zone, can be used in various medical and surgical procedures. Generally, cryogenic surgical instruments can be used to apply extremely cold temperatures to a target tissue of a patient. Cryogenic surgical instruments can be used for cryoablation and/or cryoanalgesia, for example. The present disclosure contemplates that some cryogenic surgical instruments can be supplied with one or more cryogenic fluids, which can be used to cool a tissue-contacting, active portion, such as an ablation tip. Some cryogenic surgical instruments can include supply conduits, which convey cryogenic fluid to the ablation tip, and exhaust conduits, which convey used cryogenic fluid away from the ablation tip. Some cryogenic surgical instruments can utilize cryogenic fluids supplied at high pressures. For example, some cryogenic surgical instruments employing Joule-Thompson expansion in the ablation tip can receive liquid nitrous oxide at up to about 1200 psi and about room temperature and/or can exhaust the nitrous oxide as a gas or mixed phase of gas and liquid at about 45 psi and about −90° F. The cryogenic probe and associated conduits and connectors can be designed to withstand such pressures and temperatures. Below the knee applications are illustrative, though aspects of the present disclosure have application to above the knee and to upper limb applications as well.

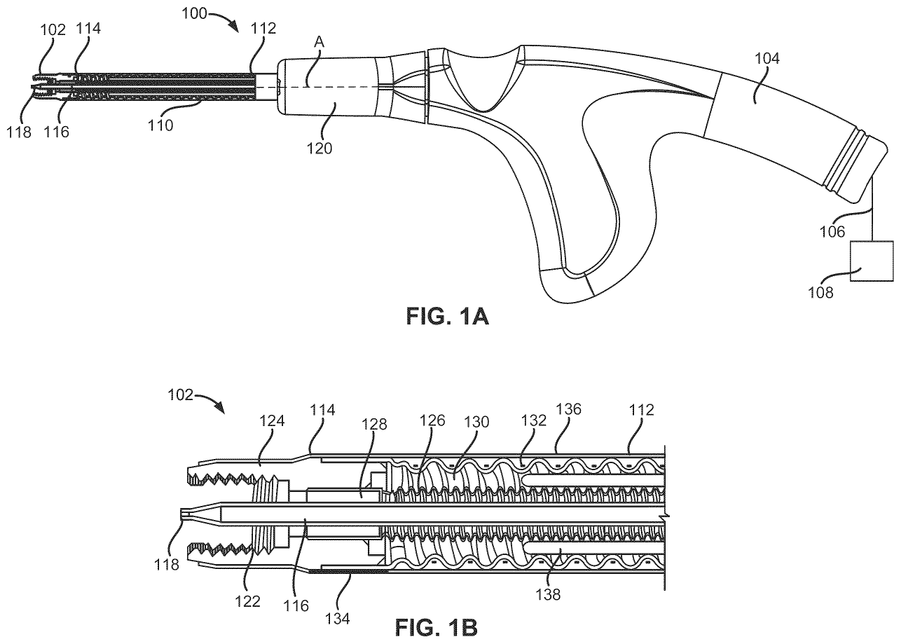

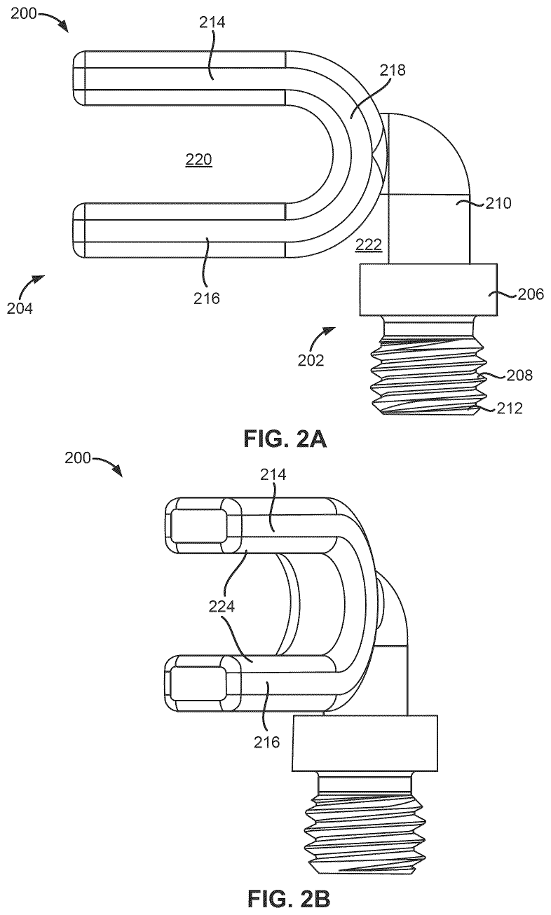

FIG. 1A illustrates a cryogenic probe 100 which can be a part of a cryogenic surgical system, which can include a cryogenic module 108. The cryogenic probe 100 can be configured to operatively couple to the cryogenic module 108, which can be configured to supply cryogenic fluid to and/or receive cryogenic fluid from the cryogenic probe 100, as well as provide monitoring and/or control functions. Example cryogenic fluids can include one or more of nitrous oxide, argon, carbon dioxide, and/or phase change fluids (e.g., liquid nitrogen). As used herein, “fluid” can refer to a substance in a liquid phase, a gas phase, a mixture of liquid and gas phases, and/or a supercritical phase.

The cryogenic probe 100 can include a handle 104, which can be configured to be grasped by a user (e.g., surgeon) and/or engaged by a robotic device (e.g., a surgical robot). More generally, the handle 104 can comprise any structure that can be configured to be secured, held, and/or manipulated to position or restrain the cryogenic probe 100, regardless of whether it can be utilized by a human (e.g., surgeon or assistant), robot, mechanical device, etc. The handle 104 can be configured to connect to the cryogenic module 108 using one or more connecting elements 106, which can include one or more fluid conduits (e.g., for supplying and/or exhausting cryogenic fluid) and/or one or more electrical conductors (e.g., wiring for thermocouples), for example. The cryogenic probe 100 can include an elongated, generally tubular shaft 110 disposed generally distally on the handle 104. The distal end portion 102 of the cryogenic probe 100 can include an end effector (not shown), which can be disposed distally on the shaft 110. The end effector can be utilized to apply extremely cold temperatures for cryogenic ablation of a target tissue 18, for example. The shaft 110 can comprise a length of about 5 inches to about 8 inches (e.g., about 7 inches) from the handle 104 to the distal end.

The shaft 110 can include a first, proximal portion 112 and/or a second, distal portion 114. In some example embodiments, the proximal portion 112 of the shaft 110 can be generally rigid and/or generally elastically deformable. As used herein, “rigid” can describe a shaft (or portion thereof) that does not deform or deforms minimally when subject to forces consistent with normal, intended use of the device. For example, the proximal portion 112 of the embodiment illustrated in FIG. 1A can be configured to retain and/or return to a generally straight shape.

The distal portion 114 of the shaft 110 can be bendable in one or more curves and/or in one or more planes. As used herein, “bendable” can describe a shaft (or portion thereof) that is elastically and/or plastically deformable in bending when subject to forces consistent with normal, intended use of the device. For example, a bendable shaft (or portion thereof) can be flexible and/or malleable. As used herein, “flexible” can describe a shaft (or portion thereof) that substantially deforms elastically and/or returns substantially to its original shape when an applied stress is removed, when subject to forces consistent with normal, intended use of the device. As used herein, “malleable” can describe a shaft (or portion thereof) that can be bent into a desired configuration and will remain substantially in that configuration when an applied stress is removed, when subject to forces consistent with normal, intended use of the device. Some example embodiments can be configured for bending by hand (e.g., without tools). Some example embodiments can be configured for bending using one or more tools, such as a manual, hand-held tubing bender tool.

The distal portion 114 and/or the proximal portion 112 of the shaft 110 can be configured to be more rigid than corresponding portions of similar devices. Accordingly, some such embodiments according to at least some aspects of the present disclosure can be capable of being utilized at more extreme angles associated with complex surgeries and/or more difficult entry angles as compared to similar devices.

The distal portion 114 of the shaft 110 can be configured for bending at an angle relative to a longitudinal axis A of the shaft 110 and/or the proximal portion 112 of the shaft 110. For example, the distal portion 114 of the shaft 110 can be bendable about 180 degrees. The distal portion 114 of the shaft 110 can be bendable in any plane that is generally parallel to the longitudinal axis A. In some embodiments, the distal portion 114 of the shaft 110 can be bendable in more than one plane. For example, the distal portion 114 of the shaft 110 can be bendable both in a plane generally parallel to the longitudinal axis A of the shaft 110 (e.g., generally upward) as well as in a plane disposed obliquely relative to the longitudinal axis A of the shaft 110 (e.g., generally leftward). As used herein, “obliquely” can refer to an angle that is neither perpendicular nor parallel. In some such embodiments, the distal portion 114 of the shaft can be bent in a plane generally orthogonal to the longitudinal axis A of the shaft 110 and/or in a plane that is inclined relative to the longitudinal axis A of the shaft 110. In some embodiments, the distal portion 114 of the shaft 110 can be bendable in two or more curves in the same or different planes, such as upward and downward (or leftward and rightward), generally in an S-shape.

Although the example embodiment illustrated in FIG. 1A can include a shaft 110 including both a generally rigid portion and a generally bendable portion, various alternative example embodiments can include shafts including only one or more generally bendable portions, only one or more generally rigid portions, or any combination of one or more generally bendable portions and/or one or more generally rigid portions, in any arrangement. In the illustrated embodiment, the generally rigid portion is generally straight; however, alternative embodiments can include generally rigid portions including one or more curves formed therein, such leftward, rightward, upward, and/or downward from the perspective of the user. In the illustrated embodiment, the generally rigid portion is fixed relative to the handle so that the generally rigid portion is not rotatable relative to the handle 104. However, in an alternate exemplary embodiment, the generally rigid portion can be rotatable relative to the handle 104.

The shaft 110 can include one or more internal conduits (e.g., which can at least partially define corresponding lumens and/or flow paths) configured to direct cryogenic fluid to and/or from the handle 104 and/or one or more electrical conductors extending to and/or from the handle 104. For example, the shaft 110 can include a supply conduit 116 configured to convey cryogenic fluid from the handle 104 to the end effector, an exhaust conduit 126 configured to convey spent cryogenic fluid from the end effector, and/or one or more wires 132 connected to a thermocouple 134. In the illustrated embodiment, the supply conduit 116 is constructed from stainless steel (e.g., 304/304L stainless steel, temper: hard), and the exhaust conduit 126 is constructed from stainless steel, though alternative materials can be used in other embodiments. The thermocouple 134 can be configured for sensing a temperature during operation of the device, such as a temperature of the distal end portion 102 of the cryogenic probe 100. In the illustrated embodiment, the thermocouple 134 can be positioned closer to the distal end than in similar cryogenic probes, thus providing more relevant temperature information than in other cryogenic probes. As used herein, “spent cryogenic fluid” can refer to cryogenic fluid that has exited the end effector (e.g., into the exhaust conduit 126), regardless of its phase, temperature, or pressure, and regardless of whether it can be capable of further cooling.

The supply conduit 116 can be generally concentrically disposed within the exhaust conduit 126. As used herein, “concentric” can describe components which are arranged so that they have a common center point and/or axis (e.g., longitudinal axis A). In the illustrated embodiment, cryogenic fluid flowing to the end effector flows within the supply conduit 116, and spent cryogenic fluid flowing from the end effector flows through the annular lumen inside of the exhaust conduit 126 and outside of the supply conduit 116. The supply conduit 116 and/or the exhaust conduit 126 can be disposed generally concentrically within other components of the shaft 110. In alternative embodiments, the conduits 116, 126 can be disposed alongside one another (e.g., generally parallel), within one another non-concentrically, and/or within the shaft 110 non-concentrically. The exhaust conduit 126 can have an exhaust conduit end portion 128 at a distal end.

The supply conduit 116 can include a nozzle 118 (e.g., an orifice) through which cryogenic fluid from the supply conduit 116 travels. In operation, cryogenic fluid supplied from the cryogenic module 108 can flow through the supply conduit 116 and the nozzle 118 into an end effector as will be described below. Generally, the cryogenic fluid exiting the nozzle 118 can be at a higher pressure upstream from the nozzle 118 and can be allowed to expand downstream of the nozzle 118 at a significantly lower pressure, thereby creating a Joule-Thompson expansion and significantly lowering the temperature of the cryogenic fluid. In some example embodiments utilizing nitrous oxide as a cryogenic fluid, the nitrous oxide can be supplied as a liquid at a temperature of about 27° C. and a pressure of about 800 psi upstream of the nozzle 118, and can comprise a gaseous phase or a mixed phase of gas and liquid at approximately 45 psi and −68° C. Alternatively, the cryogenic fluid can be supplied as a supercritical fluid. As Joule-Thompson expansion continues occurring within the end effector, the wall 122 becomes cooled enough for use in a cryosurgical procedure (e.g., cryoablation). By way of example, depending upon the cryogenic fluid utilized and the state of the fluid, exemplary flow rates for cryogenic fluid through the nozzle 118 can range between approximately fifteen to greater than one hundred cubic centimeters per minute.

The shaft 110 can include an insulating portion radially interposing the conduits 116, 126 and an outer surface 136 of the shaft 110. In some example embodiments, the shaft 110 can be configured to provide greater insulating capability than corresponding portions of similar devices, thus providing operational advantages.

The insulating portion can include a vacuum insulating layer 130 radially outward from the exhaust conduit 126. As used herein, “vacuum insulating layer” refers to an at least partially evacuated volume configured to reduce heat transfer, and includes a near vacuum, a partial vacuum, and a total vacuum. In the illustrated embodiment, the vacuum insulating layer 130 comprises a generally annular evacuated volume at least partially enclosed by an outer surface 136 of the exhaust conduit 126.

A thermal barrier element 138 can be disposed within the vacuum insulating layer 130, such as radially between the exhaust conduit 126 and the outer surface 136. For example, the thermal barrier element 138 can comprise a generally tubular, woven ceramic material. In alternative embodiments, the thermal barrier element 138 can comprise other insulative materials capable of withstanding both cryogenic temperatures and brazing temperatures, such as fiberglass, elastomers, foams, silicon, carbon fiber, and composites. In some alternative embodiments without vacuum insulation, or in the illustrated embodiment if the integrity of the evacuated volume is compromised, the thermal barrier element 138 can be configured to provide thermal insulation sufficient to allow the device to meet desired performance requirements pertaining to shaft temperature.

The shaft 110 can be coiled and can be configured to provide the desired rigidity in the proximal portion 112 and the desired bendability in the distal portion 114 by utilizing a substantially straight-walled construction in the proximal portion 112 and a substantially convoluted construction in the distal portion 114. As used herein, “convoluted” refers to an object having a form or shape that is not smooth and can be folded in curved or tortuous windings, and includes corrugations, whether or not the corrugations are spiral or otherwise. In particular, in this embodiment, in the proximal portion 112, the supply conduit 116, the exhaust conduit 126, and the shell are each formed as a substantially straight walled (e.g., non-convoluted) tube. In the distal portion 114, the exhaust conduit 126 is formed as a substantially convoluted tube. In the illustrated embodiment, the convolutions are spiraled and/or helical in shape; however, other convolutions, such as circumferential corrugations, can be utilized in alternative embodiments. In some example embodiments, one or more wires 132 connected to the thermocouple 134 can be routed along the distal portion 114 of the shaft 110 in a generally helical manner, such as wrapped around the distal portion 114 within the generally helical grooves. In some alternative embodiments, the supply conduit 116, the exhaust conduit 126, and/or the shell can include convolutions in the proximal portion 112 of the shaft 110.

Alternatively, or in addition, the illustrative cryogenic surgical instrument can include cryogenic cooling aspects disclosed in U.S. Pat. No. 11,628,007 B2 to Skorich et al., entitled “Cryogenic probe” and issued on 18 Apr. 2023, the disclosure of which is hereby incorporated by reference in its entirety. Example cryogenic fluids can include one or more of nitrous oxide, argon, carbon dioxide, and/or phase change fluids (e.g., liquid nitrogen). As used herein, “fluid” can refer to a substance in a liquid phase, a gas phase, and/or a mixture of liquid and gas phases.

Referring to FIG. 1B, cryogenic fluid supplied from the cryogenic module 108 can flow through the supply conduit 116 and the nozzle 118 and into the end effector (not shown). Generally, the cryogenic fluid exiting the nozzle 118 can be at a higher pressure upstream from the nozzle 118 and can be allowed to expand downstream of the nozzle 118 at a significantly lower pressure, thereby creating a Joule-Thompson expansion and significantly lowering the temperature of the cryogenic fluid. In some example embodiments utilizing nitrous oxide as a cryogenic fluid, the nitrous oxide can be supplied as a liquid at a temperature of about 27° C. and a pressure of about 800 psi upstream of the nozzle 118, and can comprise a gaseous phase or a mixed phase of gas and liquid at approximately 45 psi and −68° C. within the end effector. Alternatively, the cryogenic fluid can be supplied as a supercritical fluid. As Joule-Thompson expansion continues occurring within the end effector, the wall 122 becomes cooled enough for use in a cryosurgical procedure (e.g., cryoablation), such as by bringing the end effector into contact with tissue to be ablated. By way of example, depending upon the cryogenic fluid utilized and the state of the fluid, exemplary flow rates for cryogenic fluid through the nozzle 118 can range between approximately fifteen to greater than one hundred cubic centimeters per minute.

The present disclosure contemplates that, during operation of the cryogenic probe 100, cryogenic fluid supplied to the end effector and/or cryogenic fluid exhausted from the end effector can cause cooling of the shaft 110. In some circumstances, cryogenic fluid flowing through the shaft 110 can be cold enough to cause undesired thermal effects, such as freezing of non-target tissue or adherence of the shaft 110 to non-target tissue. For example, during procedures performed in the chest, it can be advantageous to avoid inadvertent freezing of or adherence to lung tissue and/or the periphery of an opening through the chest wall. Accordingly, in the illustrated embodiment, the shaft 110 includes an insulating portion radially interposing the conduits 116, 126 and an outer surface 136 of the shaft 110. In some example embodiments, the shaft 110 can be configured to provide greater insulating capability than corresponding portions of similar devices, thus providing operational advantages.

In the illustrated embodiment, the insulating portion can include a vacuum insulating layer 130 radially outward from the exhaust conduit 126. As used herein, “vacuum insulating layer” refers to an at least partially evacuated volume configured to reduce heat transfer, and includes a near vacuum, a partial vacuum, and a total vacuum. In the illustrated embodiment, the vacuum insulating layer 130 comprises a generally annular evacuated volume at least partially enclosed by an outer surface of the exhaust conduit 126.

In the illustrated embodiment, a thermal barrier element 138 is disposed within the vacuum insulating layer 130. For example, the thermal barrier element 138 can comprise a generally tubular, woven ceramic material. In alternative embodiments, the thermal barrier element 138 can comprise other insulative materials capable of withstanding both cryogenic temperatures and brazing temperatures, such as fiberglass, elastomers, foams, silicon, carbon fiber, and composites. In some alternative embodiments without vacuum insulation, or in the illustrated embodiment if the integrity of the evacuated volume is compromised, the thermal barrier element 138 can be configured to provide thermal insulation sufficient to allow the device to meet desired performance requirements pertaining to shaft temperature.

In the illustrated embodiment, the shaft 110 is configured to provide the desired rigidity in the proximal portion 112 and the desired bendability in the distal portion 114 by utilizing a substantially straight-walled construction in the proximal portion 112 and a substantially convoluted construction in the distal portion 114. As used herein, “convoluted” refers to an object having a form or shape that is not smooth and can be folded in curved or tortuous windings, and includes corrugations, whether or not the corrugations are spiral or otherwise. In particular, in this embodiment, in the proximal portion 112, the supply conduit 116 and the exhaust conduit 126 are each formed as a substantially straight walled (e.g., non-convoluted) tube. In the distal portion 114, the exhaust conduit 126 and the shell are each formed as a substantially convoluted tube. In the illustrated embodiment, the convolutions are spiraled and/or helical in shape; however, other convolutions such as circumferential corrugations can be utilized in alternative embodiments. In some example embodiments, one or more wires 132 connected to the thermocouple 134 can be routed along the distal portion 114 of the shaft 110 in a generally helical manner, such as wrapped around the distal portion 114 within the generally helical grooves. In some alternative embodiments, the supply conduit 116 and the exhaust conduit 126 can include convolutions in the generally rigid, proximal portion 112 of the shaft 110.

One technical problem encountered by the inventors is creating a fixture to provide a repeatable and consistent placement of a thermocouple on tissue in relation to the end effector of the device. It would be beneficial for methods of tracking tissue temperature to be repeatable and consistent to measure the temperature of the tissue at the same point in relation to the end effector of a device. One technical solution discovered by the inventors is creating a fixture that grants the ability to control the placement of tissue and thermocouples around the end effector of a device to provide tissue temperature over time. The fixture grants the ability to consistently and repeatably place thermocouples into tissue engaged with the prongs of the end effector using hollow needles to guide the thermocouples into place.

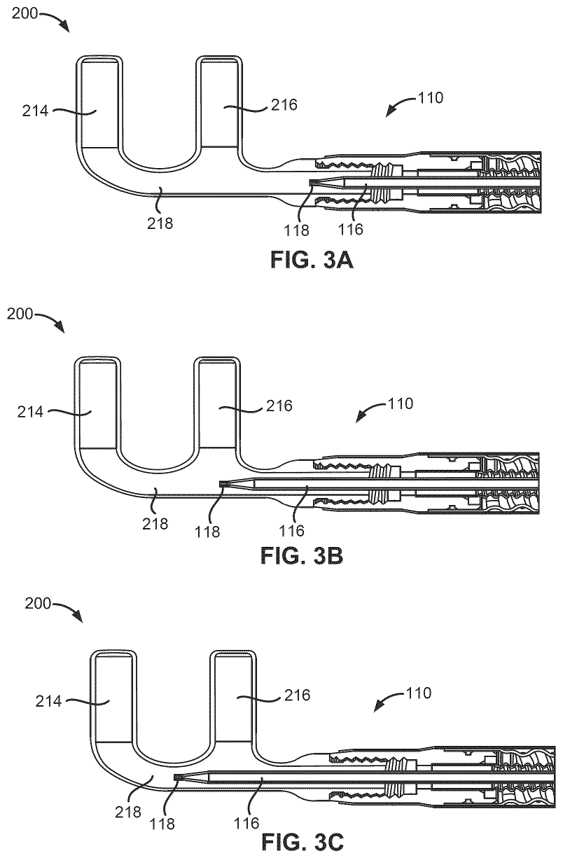

FIGS. 2A and 2B illustrate an end effector 200 for use with the cryogenic probe 100. The end effector 200 can comprise a base portion 202 and a head portion 204. The base portion 202 can comprise a threaded portion 206, a flanged portion 208, and a shaft portion 210. The threaded portion 206 can comprise a plurality of helical screw threads 212 to be inserted into the shaft 110 such that the threads 212 couple to threads within the wall 122 of the shaft 110.

In some variations, the end effector 200 can snap, slip, or fasten into the shaft 110. In other variations, the end effector 200 can be manufactured by metal injection molding, 3-D printing, or a combination thereof. In other variations, the end effector 200 can be made monolithic or in separate pieces. The end effector 200 can be made of stainless steel, aluminum, copper with gold over nickel plating, titanium, gold-plated copper, copper, alloys of the above-mentioned materials, or a combination thereof.

The head portion 204 can comprise a first prong 214, a second prong 216 spaced apart from the first prong 214, and a curved section 218 therebetween. The first prong 214 and the second prong 216 can extend from the curved section 218 in a direction perpendicular to a longitudinal axis of the shaft 110. The first prong 214 and the second prong 216 can be spaced apart along the longitudinal axis, defining a gap 220 for nerves or tissue to be placed. It should be appreciated that the prongs 214, 216, and curved section 218 can extend from the shaft 110 at various angles.

The curved section 218 can also extend in the direction perpendicular to a longitudinal axis of the shaft 110. A space 222 can be defined between the curved section 218 and the base portion 202. The space 222 can facilitate nerve grasping as desired by the user.

In other variations, the prongs 214, 216 can be slanted inward toward the curved section 218. In other variations, a first portion of the prongs 214, 216 can be slanted inward toward the curved section 218 such that a second portion of the prongs 214, 216 are perpendicular to the longitudinal axis of the shaft 110.

The end effector 200 can comprise a hollow interior throughout to receive cryofluid (e.g., liquid nitrogen) from the supply conduit 116. In some embodiments, gas can be used (e.g., carbon dioxide, nitrous oxide, argon). The end effector 200 can comprise a total length of about 0.8 inches to about 1.2 inches (e.g., about 0.963 inches). The end effector 200 can comprise a maximum width of about 0.2 inches to about 0.3 inches (e.g., about 0.225 inches). The first prong 214 and the second prong 216 can each comprise a diameter of about 0.1 inch to about 0.2 inches (e.g., about 0.15 inches). The end effector 200 can comprise a wall thickness of about 0.01 inches to about 0.02 inches (e.g., about 0.015 inches) to promote a uniform freeze around the end effector 200. In other variations, the wall thickness can vary throughout the cross-section of the end effector 200.

The distance between the central axis of the first prong 214 and the central axis of the second prong 216 can be about 0.35 inches to about 0.45 inches (e.g., about 0.39 inches). The gap between the prongs 214, 216 can be about 0.15 inches to about 0.2 inches (e.g., about 0.18 inches). The length of the end effector 200 from the curved section 218 to the distal end of the prongs 214, 216 can be about 0.5 inches to about 0.6 inches (e.g., about 0.55 inches).

As seen in FIG. 2B, a cross-section of the first prong 214 and the second prong 216 can be substantially rectangular. In some variations, the cross-section can be circular, oval-shaped, square, or any combination thereof. The edges 224 of the prongs 214, 216 can comprise tapered surfaces such that the edges 224 do not damage tissue when the end effector 200 is advanced to a target site. The prongs 214, 216 can comprise a consistent cross-section along their lengths in order to provide consistent gas flow during the procedure.

The cross-section of the prongs 214, 216 can comprise a length of about 0.15 inches and a width of about 0.1 inches. The dimensions of the prongs 214, 216 are such that the smallest (about 1 mm) and largest (about 27 mm) nerves in the legs can be in contact with the end effector 200 for freezing during amputation procedures.

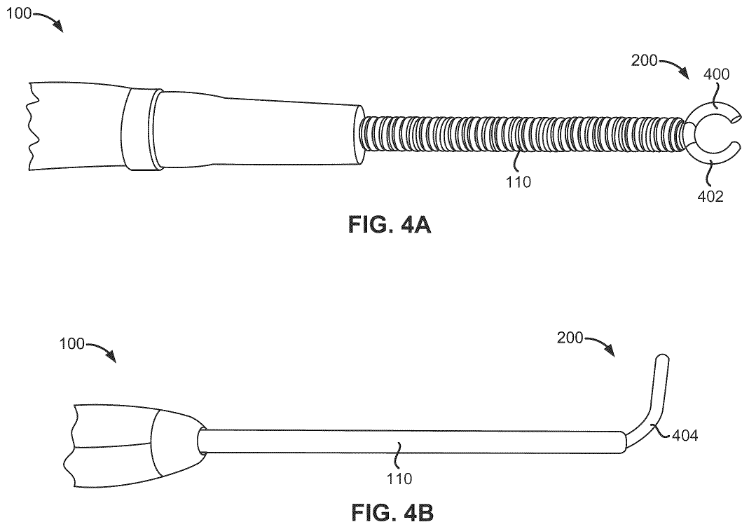

FIG. 3A illustrates a side view of the end effector 200 and the shaft 110 comprising the nozzle 118. The nozzle 118 can provide cryofluid to the end effector 200 at a controlled rate. The nozzle 118 can be positioned in various locations along the length of the shaft 110. For example, the distal end of the nozzle 118 can be positioned about 0.02 inches from the distal end of the shaft 110.

As seen in FIG. 3B, the distal end of the nozzle 118 can be positioned aligned with the first prong 214 and proximal to the curved section 218, about 0.278 inches from the distal end of the shaft 110. As seen in FIG. 3C, the nozzle 118 can be positioned at the curved section 218 of the end effector 200, about 0.513 inches from the distal end of the shaft 110. To extend the nozzle 118 position, the length of the shaft 110 and corresponding components can be adjusted accordingly. Both the location of the nozzle 118 and the flow rate of the cyrofluid can affect the flow of the cryofluid into the prongs 214, 216, affecting the consistency of the procedure. The nozzle 118 can comprise an orifice resulting in a cone jet or a helix nozzle resulting in a spiral jet.

In some variations, two or more nozzles can be used to direct cryofluid into the end effector 200. In some variations, the nozzle 118 can be bent into the prongs 214, 216 to direct flow into the prongs 214, 216. In some variations, the walls of the end effector 200 can be reinforced with baffles for thicker sections to promote cooling at certain areas of the end effector. In some variations, tracks can be placed within the prongs 214, 216 to direct flow within. In some variations, a nozzle 118 with multiple orifices can be provided to direct flow into the prongs 214, 216.

The end effector 200 can be configured to contact one or more of the following nerves or groups of nerves: saphenous nerve, deep fibular nerve, sciatic nerve, deep fibular bundle, superficial fibular nerve, tibial nerve, tibial bundle, medial sural cutaneous nerve, lateral sural cutaneous nerve, lateral communicating branch, and superficial peroneal nerve.

The end effector 200 can be used to transmurally freeze nerves or tissue of various sizes and locations in a robust and efficient manner (i.e., within 60 seconds). Freezing can be achieved as proximally as possible such that the nerve end(s) can be cut, then retracted to be protected by tissue in the amputation stump.

In some methods, the first prong 214 and the second prong 216 of the end effector 200 can be rotated by the user to isolate nerves from surrounding structures and freeze multiple nerves at different angles and while the user is in different positions and/or orientations around the operating table.

In some methods, the nerve can be woven through in a zig-zag pattern between the first prong 214 and the second prong 216 to stabilize the nerve and position the end effector 200 for cryoablation such that a continuous freeze of a known length is achieved wherein the regrowth of the axial nerve will be incomplete along that length. The cut can be performed at the distal end of the ice ball. In other variations, the nerve(s) can be placed over and/or hooked around the curved section 218 of the end effector, between the first prong 214 and the second prong 216. In other variations, the nerve(s) can be twisted or wrapped around one of the prongs. In other variations, the nerve(s) can be placed across the prongs 214, 216 such that the user can pull the end effector 200 towards the user to manipulate the nerves. In other variations, the nerve(s) can be pressed by the end effector 200 from the anterior surface of the nerves to manipulate positioning of the nerves. Accordingly, the cryogenic probe 100 can allow various applications of force applied to nerves (i.e., the user can apply constant or intermittent pressure).

The surfaces of the first prong 214 and the second prong 216 can provide for nerve contact, and accordingly, can engage various nerves that range in diameter, for example, between 1 mm to 27 mm as is present in lower limb nerves. As such, the user can maintain close control of the end effector 200 during cryoablation due to the control of the nerve(s) via the prongs 214, 216.

In other variations, the end effector 200 can comprise prongs that clamp tissue before freezing. In other variations, the end effector 200 can comprise a cutting element on the prongs to dissect tissue.

In other variations, the end effector 200 can be used for lobed structures, blood vessels, solid organs, or other locations within the body (e.g., upper extremities). The cryogenic probe 100 can freeze nerves before or after the nerves or the limbs are cut, with or without added visualization.

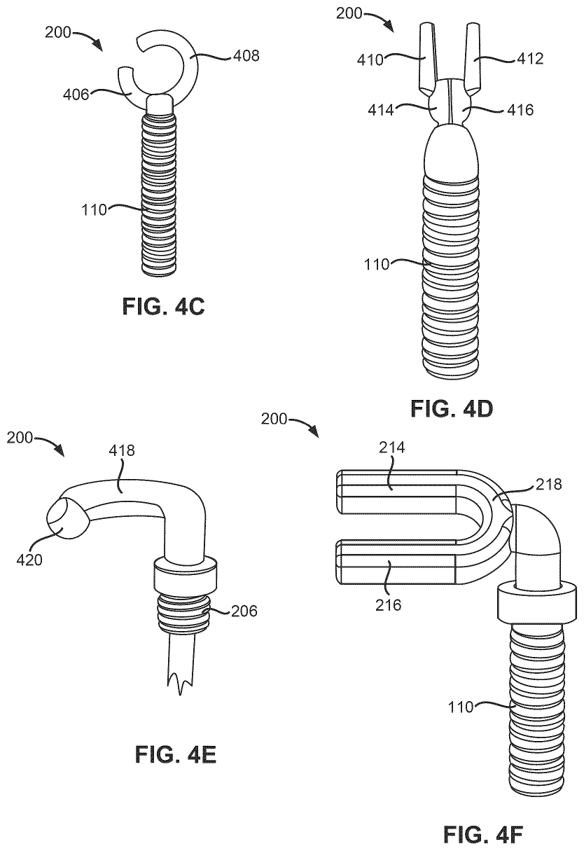

FIGS. 4A-F illustrate variations of end effectors which can be used with the cryogenic probe 100. FIG. 4A illustrates an end effector 200 comprising two curved prongs 400, 402 that extend symmetrically and terminate to form a C-shape. FIG. 4B illustrates an end effector 200 comprising a single prong 404 that extends 90 degrees from the shaft 110.

FIG. 4C illustrates an end effector 200 comprising two curved prongs. A first curved prong 406 is longer than a second curved prong 408 to form an opening between the prongs at a location offset from the longitudinal axis of the shaft 110.

FIG. 4D illustrates an end effector 200 comprising prongs 410, 412 parallel to the longitudinal axis of the shaft 110. The prongs 410, 412 can optionally comprise base portions 414, 416 that couple the prongs 410, 412 to the shaft 110. FIG. 4E illustrates an end effector 200 comprising a prong 418 shaped as a hook that comprises a semi-circular shape at end section 420 extending perpendicular to the longitudinal axis of the shaft. FIG. 4F illustrates an end effector 200 comprising a curved section 218, first prong 214, and second prong 216 extending about 45 degrees from a first plane perpendicular to the longitudinal axis of the shaft 110 and about 90 degrees from a second plane perpendicular to the longitudinal axis of the shaft 110.

FIG. 4G illustrates an end effector 200 comprising a curved section 218, first prong 214, and second prong 216 extending about 45 degrees from the longitudinal axis of the shaft 110.

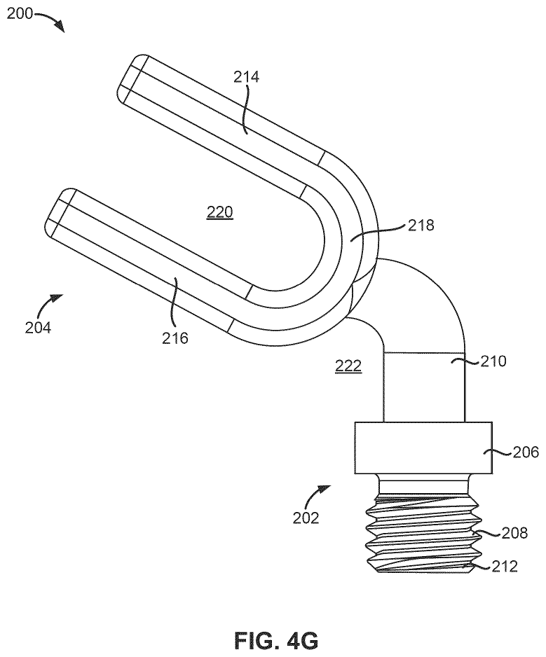

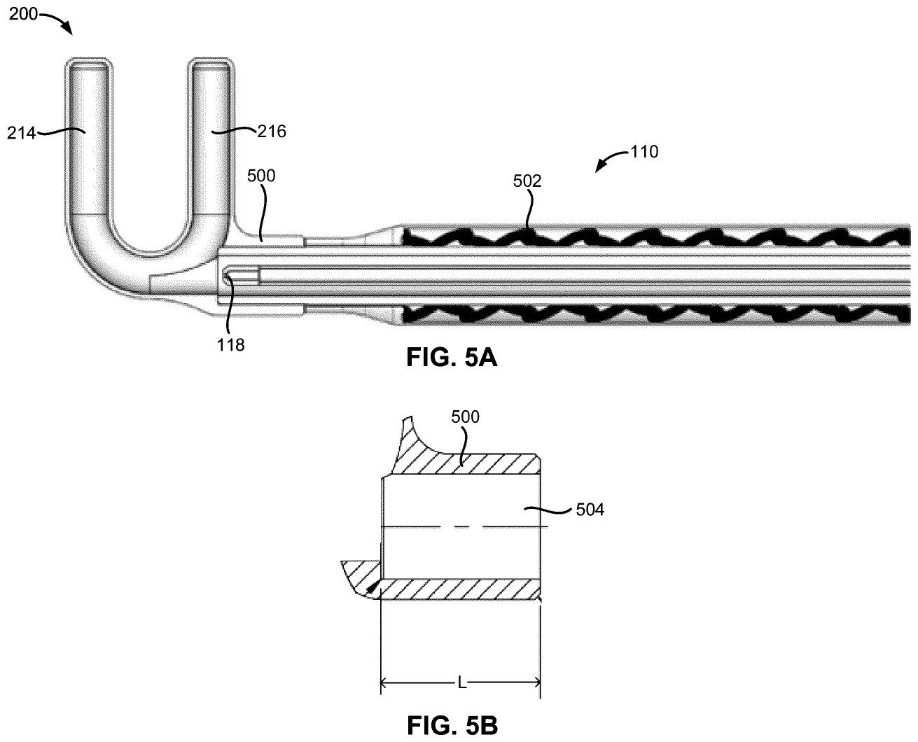

FIG. 5A illustrates another variation of the end effector 200 having a nozzle 118 that extends to a longitudinal distance at which the second prong 216 is located. The nozzle 118 extends to a distance such that fluid delivered through the nozzle 118 is delivered uniformly to both the first prong 214 and the second prong 216 at appropriate temperatures (e.g., about 40° C.).

As seen in FIGS. 5A and 5B, the end effector 200 can comprise a prong base 500 that terminates at the second prong 216, maintaining a constant outer diameter from the proximal end to the distal end. Accordingly, this variation allows for the prong base 500 and the shaft 110 to be used without cutting the shaft 110 down to fit within a longer neck portion. The diameter of each of the first prong 214 and the second prong 216 can be about 0.15 inches. The variation shown in FIG. 5A comprises no (or minimal) neck portion between the prong base and the second prong compared to the embodiments in FIGS. 3A to 3C.

The prong base 500 can comprise a cavity 504 in which the shaft 110 can be inserted therein. The prong base 500 can have a diameter of about 2.55 inches. The cavity can have a width of about 0.12 inches to about 0.185 inches. The prong base 500 can comprise a cavity length L of about 0.28 inches. The entire width of the end effector 200 can be about 0.823 inches.

The nozzle 118 can be spaced from a center line of the second prong 216 by about 0.03 inches. The nozzle 118 tip can be spaced from a distal end of the shaft 110 by about 0.01 inches.

The end effector 200 can be used to target both exposed nerves and in situ nerves. In some variations, the end effector 200 can be used in redo or revision procedures. For example, during post-amputation, the residual stump is surgically reopened to manage nerves or other potential sources of pain such as bone spurs or infectious tissue. Access for the latter can be as open as an initial/index amputation.

The first prong 214 and the second prong 216 can be cylindrical in order to conform to nerves as well as provide for a smooth surface when the device is navigating the anatomy.

In this variation, the shaft 110 can comprise a braided insulation 502 that provides for electrical insulation of the shaft 110 while providing mechanical strength and flexibility for the device. The braided insulation 502 can be plastic.

In some variations, the shaft 110 can comprise a thread that couples to an inner thread of the end effector 200. In other variations, the shaft 110 can be welded onto the end effector 200. In other variations, an adhesive (e.g., epoxy) can be used to join the shaft 110 and the end effector 200. In other variations, the end effector 200 can snap onto the shaft 110.

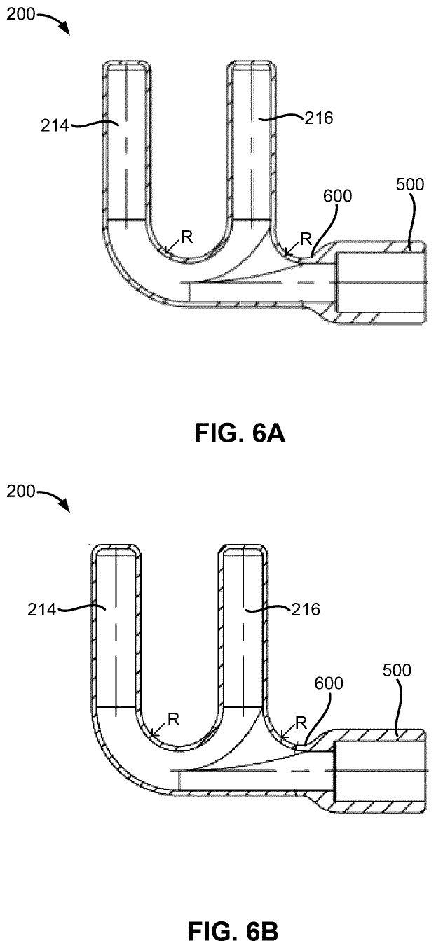

FIGS. 6A and 6B illustrate a variation of the end effector wherein the end effector 200 comprises a neck portion 600 between the second prong 216 and the prong base 500. In this variation, the neck portion 600 can be proximal to the second prong 216, creating an outer diameter at the neck portion 600 less than an outer diameter of the remainder of the prong base 500.

In some variations, the radius R of the prong leading into the first prong 214 and the second prong 216 can be unequal, as seen in FIG. 6A. In other variations, the radius R of the prong leading into the first prong 214 and the second prong 216 can be equal, as seen in FIG. 6B. The radius leading into each prong can impact and facilitate nerve grasping without drastic arm or wrist movement from the user.

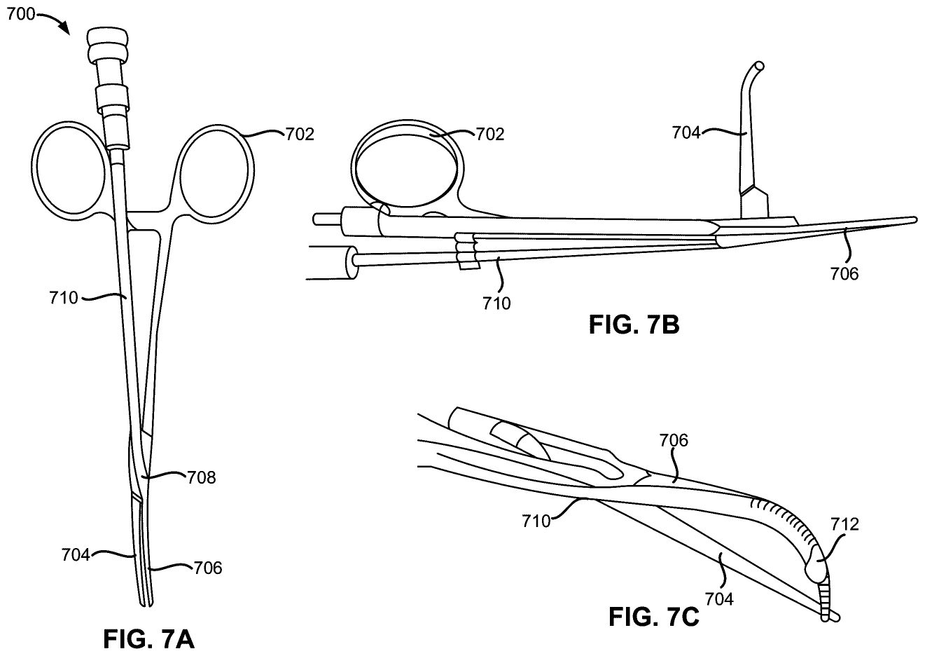

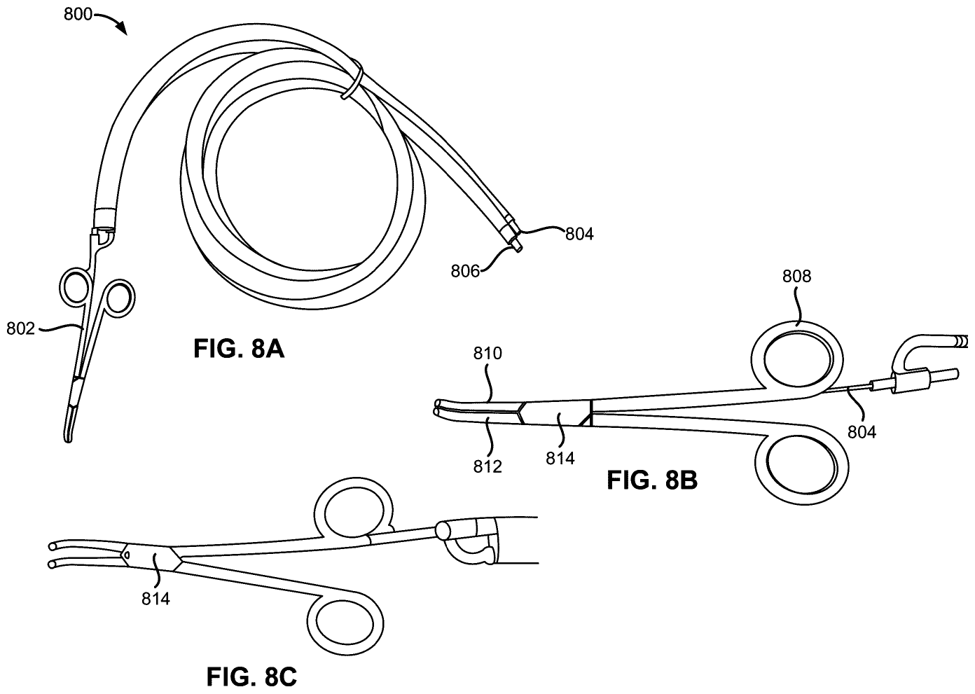

The cryogenic surgical instrument depicted in FIGS. 7A to 8C can include cryogenic cooling aspects similar to the “cryoICE BOX” cryogenic surgical unit available from AtriCure, Inc. of Mason, Ohio, or may include one or more components providing similar functionality. Alternatively, or in addition, the illustrative cryogenic surgical instrument may include cryogenic cooling aspects disclosed in U.S. Pat. No. 11,628,007 B2 to Skorich et al., entitled “Cryoprobe” and issued on 18 Apr. 2023, the disclosure of which is hereby incorporated by reference in its entirety. Example cryogenic fluids may include one or more of nitrous oxide, argon, carbon dioxide, and/or phase change fluids (e.g., liquid nitrogen). As used herein, “fluid” may refer to a substance in a liquid phase, a gas phase, and/or a mixture of liquid and gas phases.

FIGS. 7A to 7C illustrate a device 700 having a handle 702, a first jaw 704, a second jaw 706, and a hinge 708. The second jaw 706 can be coupled to a cryogenic probe 710. In this variation, the second jaw 706 can be cored out and the cryogenic probe 710 can be coupled to the second jaw 706. The first jaw 704 can be a non-freezing jaw. The first jaw 704 and the second jaw 706 can both comprise epoxy 712 on the outside surfaces of the jaws. In this variation, the flow of the device can be about 430 ccm and the power needed to run the device can be about 11.6 W. The ice ball created by the cryogenic energy can be about 6 mm in width.

The method can comprise an air freezing method or a water bath freezing method for cooling the cryogenic probe 710. The freezing can be applied throughout the one or more nerves and surrounding tissue accordingly as the end effector is positioned across the target site. In some variations, the one or more nerves can be manipulated by the jaws such that parts of the nerve receive more cryogenic energy than others. In some variations, the water freezing method can be applied to the second jaw 706 or both the first jaw 704 and the second jaw 706.

FIGS. 8A to 8C illustrate a system 800 having a device 802, an inlet tube 804, and an exhaust tube 806. The device 802 can comprise a handle 808, a first jaw 810, a second jaw 812, and a hinge 814. A lumen can be created within the first jaw 810 to create a space for a cryogenic inlet line 804 to be placed within. The second jaw 812 can then be used as a freezing jaw. The cryogenic inlet tube 804 can be coupled to a cryogenic exhaust source (not shown).

In this variation, the flow of the device can be about 314 ccm and the power needed to run the device can be about 7.03 W. The ice ball created by the cryogenic energy can be about 5 mm in width.

Methods and devices herein can be used to clamp over, under, or to the side of the one or more nerves at the end effector and slide proximally to engage the end effector with the one or more nerves. The end effector can then hook underneath the one or more nerves and can press against the one or more nerves from the anterior surface of the nerves.

Unless specifically indicated, it will be understood that the description of the structure, function, and/or methodology with respect to any illustrative embodiment herein can apply to any other illustrative embodiments. More generally, it is within the scope of the present disclosure to utilize any one or more features of any one or more example embodiments described herein in connection with any other one or more features of any other one or more other example embodiments described herein. Accordingly, any combination of any of the features or embodiments described herein is within the scope of this disclosure.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes can be made without departing from the scope of the disclosure. Likewise, it is to be understood that it is not necessary to meet any or all of the identified advantages or objects disclosed herein in order to fall within the scope of the disclosure, since inherent and/or unforeseen advantages can exist even though they have not been explicitly discussed herein.

While the innovation has been described with reference to exemplary embodiments, it will be understood by those skilled in the art that various changes can be made, and equivalents can be substituted for elements thereof without departing from the scope of the innovation. In addition, many modifications can be made to adapt a particular system, device, or component thereof to the teachings of the innovation without departing from the essential scope thereof. Therefore, it is intended that the innovation not be limited to the particular embodiments disclosed for carrying out this innovation, but that the innovation will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the innovation. As used herein, the singular forms “a”, “an” and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present innovation has been presented for purposes of illustration and description but is not intended to be exhaustive or limited to the innovation in the form disclosed. Many modifications and variations will be apparent to those of ordinary skill in the art without departing from the scope and spirit of the innovation. The embodiments were chosen and described in order to best explain the principles of the innovation and the practical application, and to enable others of ordinary skill in the art to understand the innovation for various embodiments with various modifications as are suited to the particular use contemplated.

What is claimed is:

1. A cryogenic probe, comprising,
a handle;
a shaft comprising a proximal end coupled to the handle, wherein the shaft defines a longitudinal axis;
an end effector coupled to a distal end of the shaft, wherein the end effector comprises a first prong, a second prong, and a curved section therebetween, wherein the first prong and the second prong are configured to receive cryogenic fluid, wherein the first prong and the second prong extend perpendicular to the longitudinal axis and are spaced apart along the longitudinal axis; and
a supply conduit configured to supply cryogenic fluid to the end effector.

2. The cryogenic probe of claim 1, further comprising an exhaust conduit configured to exhaust cryogenic fluid from the end effector.

3. The cryogenic probe of claim 2, wherein the exhaust conduit is concentrically disposed around the supply conduit.

4. The cryogenic probe of claim 1, further comprising a vacuum insulating layer disposed around the supply conduit.

5. The cryogenic probe of claim 4, further comprising a tubular thermal barrier element disposed within the vacuum insulating layer.

6. The cryogenic probe of claim 5, wherein the thermal barrier element is constructed of at least one of a ceramic, a fiberglass, an elastomer, a foam, silicon, carbon fiber, and a composite.

7. The cryogenic probe of claim 1, wherein the end effector is inserted at the distal end of the shaft.

8. The cryogenic probe of claim 7, wherein the end effector is coupled to the distal end of the shaft via a screw thread.

9. The cryogenic probe of claim 1, further comprising a nozzle at a distal end of the supply conduit.

10. The cryogenic probe of claim 9, wherein the nozzle terminates proximal to the curved section.

11. The cryogenic probe of claim 1, wherein the end effector comprises a prong base, wherein the prong base comprises a constant outer diameter that transitions to the second prong.

12. The cryogenic probe of claim 1, wherein a cross-section of the first prong and a cross-section of the second prong are rectangular.

13. A method for freezing one or more nerves of a patient, the method comprising:
advancing a cryogenic probe to a target site, the cryogenic probe comprising a handle, a shaft comprising a proximal end coupled to the handle, and an end effector coupled to a distal end of the shaft, wherein the end effector comprises a first prong, a second prong, and a curved section therebetween, wherein the first prong and the second prong are configured to receive cryogenic fluid, wherein the first prong and the second prong extend perpendicular to a longitudinal axis of the shaft and are spaced apart along the longitudinal axis;
positioning the end effector to grasp one or more nerves;
supplying cryogenic fluid from a supply conduit to the end effector; and
freezing the one or more nerves by applying the end effector to surface of the one or more nerves.

14. The method of claim 13, further comprising exhausting cryogenic fluid from the end effector via an exhaust conduit.

15. The method of claim 13, further comprising a nozzle within the supply conduit.

16. The method of claim 15, wherein the nozzle terminates at the curved section.

17. The method of claim 15, wherein the nozzle terminates proximal to the curved section.

18. A medical device, comprising,
a handle;
a shaft comprising a proximal end coupled to the handle, wherein the shaft defines a longitudinal axis;
an end effector coupled to a distal end of the shaft, wherein the end effector comprises a first prong, a second prong, and a prong base, wherein the first prong and the second prong are configured to receive fluid, wherein the first prong and the second prong extend at an angle with respect to the longitudinal axis, wherein the prong base comprises a cavity to receive the shaft; and
a supply conduit configured to supply fluid to the end effector.

19. The medical device of claim 18, wherein the fluid is a cryogenic fluid.

20. The medical device of claim 18, further comprising a nozzle at a distal end of the supply conduit.
